(19) United States
(12) Patent Application Publication
(10) Pub. No.: US 2013/0052639 A1
(43) Pub. Date: Feb. 28, 2013

(54) METHOD OF ANALYZING XPG ENDONUCLEASE ACTIVITY

(71) Applicants: Young Rok Seo, Seoul (KR); Koedrith Preeyaporn, Seoul (KR); Hye Lim Kim, Seoul (KR); Sang Min Lee, Seoul (KR)

(72) Inventors: Young Rok Seo, Seoul (KR); Koedrith Preeyaporn, Seoul (KR); Hye Lim Kim, Seoul (KR); Sang Min Lee, Seoul (KR)

(73) Assignee: DONGGUK UNIVERSITY INDUSTRY-ACADEMIC COOPERATION FOUNDATION, Seoul (KR)

(21) Appl. No.: 13/587,172

(22) Filed: Aug. 16, 2012

(30) Foreign Application Priority Data
Aug. 26, 2011 (KR) 10-2011-0085656
Aug. 10, 2012 (KR) 10-2012-0087800

(51) Int. Cl. C12Q 1/68; G01N 21/76; G01N 21/64

(52) U.S. Cl. 435/6.1

(57) ABSTRACT

A method of quantitatively analyzing an XPG endonuclease activity is provided. The XPG endonuclease activity can be simply and cheaply analyzed without undergoing overexpression or purification of a recombinant protein.

### CROSS-REFERENCE TO RELATED APPLICATIONS

This application claims priority to and the benefit of Korean Patent Applications Nos. 2011-0085656 and 2012-0087800, filed on Aug. 26, 2011 and Aug. 10, 2012, respectively, and the disclosures of which are incorporated herein by reference in their entirety.

### INCORPORATION BY REFERENCE

Incorporated by reference herein in its entirety is the Sequence Listing, entitled “Sequence Listing_ST25.txt,” which was created Aug. 16, 2012, size 1 kilobyte.

### BACKGROUND

1. Field of the Invention

The present invention relates to a method of analyzing an XPG endonuclease activity.

2. Discussion of Related Art

A nucleotide excision repair (NER) pathway has been considered to be a main DNA recovery system to remove chemical addition products having a high volume and UV photolysis products. In the case of humans, the deficiency of XPG (xeroderma pigmentosum (XP) of the complementation group G) that is a main enzyme in the NER pathway causes generation of cancer-prone syndromes and lots of skin cancers according to the acute UV sensitiveness. This indicates that an XPG protein plays an important role in DNA recovery and thus in maintenance of genetic stability.

The XPG protein is structure-specific endonuclease that incises a substrate having defined polarity during the nucleotide excision repair (NER). In typical in vitro studies on the catalytic activities of the purified human XPG, it was found that an XPG-like substrate has an artificial DNA structure (i.e., bubble, splayed arm, stem-loop, flap substrate) (A. O'Donovan, A. A. Davies, J. G. Moggs, S. C. West, and R. D. Wood, Nature 371 (1994a) 432-435; E. Evans, J. Fellow, A. Coffer, and R. D. Wood, EMBO. 16 (1997) 625-638). The XPG protein has a molecular weight of 135 kDa and belongs to a group of structure-specific Fent (Flap endonuclease 1). In the N terminus and an internal domain, XPG shares homologous sequences with another group of nucleases including bacteriophage T4 RNase H. Conserved acidic residues are present in an active domain of RNase H that can chelate two enzyme magnesium (Mg) ions. The same acidic residues in XPG as the conserved acidic residues may be associated with hydrolysis, and thus may be associated with an XPG-DNA bond.

During the NER, a series of biochemical procedures mediate the recognition of damaged bases, the incision of approximately 26 to 30 nucleotides, the removal of a damaged patch, and the filling of a gap, and the ligation. In an incision step, the dual incision of opposite poles in the vicinity of a junction of unpaired dual strand DNA is catalyzed by two different kinds of endonuclease. In the case of humans, an XPG protein and an ERCC1-XPF complex mediate 3′- and 5′-incisions for the dual incision, respectively. Both of the two endonucleases show structural specificity with respect to a model DNA substrate.

The 3′- and 5′-termini of a damaged DNA strand are dually incised by the XPG protein and the ERCC1-XPF complex, respectively. Meanwhile, when a typical 5′-terminus oligolabeling is applied, the 5′-terminus transferring activity of the ERCC1-XPF complex shades the 3′-incision activity of XPG. Therefore, as another approach to solve these problems, a 3′-terminus oligolabeling method using an intracellular protein extract may be considered. When this method is used, it is unnecessary to overexpress and purify target XPG as a precondition for XPG assay.

The present inventors have conducted research to develop a method of analyzing an XPG endonuclease activity with a simple and accurate manner, all of which are applicable to cells and tissues. Therefore, the present invention has been completed based on the facts.

### SUMMARY OF THE INVENTION

An object of the present invention is to provide a novel method of analyzing an XPG endonuclease activity.

Another object of the present invention is to provide a kit for analyzing an XPG endonuclease activity.

One aspect of the present invention provides a method of analyzing an XPG endonuclease activity. Here, the method includes preparing a biological extract sample including XPG (xeroderma pigmentosum (XP) of the complementation group G), preparing a DNA bubble substrate, forming the DNA bubble substrate whose 3′-terminus is labeled by attaching a detectable label to the 3′-terminus of the DNA bubble substrate, and mixing the biological extract sample and the DNA bubble substrate whose 3′-terminus is labeled.

The biological extract sample may be a cellular nuclear extract, a total cellular protein extract or a total tissue protein extract.

The DNA bubble substrate may have oligonucleotides set forth in SEQ ID NO: 1 and SEQ ID NO: 2 which complementarily bind to each other.

The detectable label may be a radioactive isotope, a fluorescent material, a luminescent material or a precursor of the luminescent material, and, more particularly, may be selected from the group consisting of ³²P, ³⁵S, ¹³¹I, ¹²³I, ¹²⁵I, ³H, carboxyfluorescein (FAM), tetramethylrhodamine (TAMRA), Cy3, Cy5, IRDye series, fluorescein, fluorescein isothiocyanate (FITC), rhodamine, Texas Red, Alexa series, digoxigenin (DIG) and biotin, but the present invention is not particularly limited thereto.

The method may further include determining whether the DNA bubble substrate is incised after the mixing of the biological extract sample and the DNA bubble substrate whose 3′-terminus is labeled.

The incised DNA bubble has a size of 30 or less nucleotides.

The mixing of the biological sample and the DNA bubble substrate may be performed in the presence of a reaction buffer.

The reaction buffer may have a pH value of 6.0 to 8.5.

The reaction buffer may include MgCl₂ or MnCl₂.

An amount of the added MgCl₂ or MnCl₂ may be in a range of 2 to 10 mM.

Another aspect of the present invention provides a kit for analyzing an XPG endonuclease activity. Here, the kit includes the biological extract and the DNA bubble substrate as described above.

The method according to the present invention may simply analyze the XPG endonuclease activity without performing additional procedures of preparing a recombinant protein to overexpress an XPG protein, separating the XPG protein and purifying the XPG protein since a biological extract including XPG other than a purified XPG protein may be directly analyzed for the endonuclease activity of XPG. Also, the analysis procedure is advantageous in that the XPG endonuclease activity can be analyzed at a level close to the actual organism activity since a protein is not easily modified during the separation and purification of the protein.

Also, the XPG endonuclease activity may be effectively evaluated using a suitable nucleotide bubble substrate, that is, a DNA bubble substrate, as the substrate of the XPG protein. In particular, to exclude an effect of a 5′-terminus incision activity of another endonuclease, ERCC1-XPF complex, which is associated with the dual incision of NER, the 3′-terminus incision activity of the XPG endonuclease may be accurately evaluated using a bubble substrate having a detectable label attached to the 3′-terminus thereof.

The biological extract sample may be a cellular nuclear protein extract, a total cellular protein extract or a total tissue protein extract. The protein extracts include XPG. When the biological extract is mixed with the DNA bubble substrate, an endonuclease, XPG, comes in contact with the DNA bubble substrate to show an incision activity.

That is, based on a bubble structure of the bubble substrate as shown in FIG. 1A, the XPG incises the 3′ terminus of the bubble structure, and the ERCC1-XPF complex (indicated by XPF) incises the 5′ terminus of the bubble structure. In general, it is known that the XPG and the ERCC1-XPF complex incise 3 to 5 bases in 3′ to 5′ and 5′ to 3′ direction from the C repeated sequence. Therefore, when the 5′-terminus-labeled bubble substrate is used as shown in FIG. 1A, the incisions by the XPG and the ERCC1-XPF complex take place at the same time. Also, even when the XPG does not have an incision activity but the ERCC1-XPF complex shows an incision activity, a labeled fragment corresponding to a size of approximately 30 bp. Therefore, it is possible to accurately analyze the XPG activity since a fragment having a size of approximately 30 bp is generated regardless of the incision activity of the XPG. On the other hand, when the 3′-terminus-labeled bubble substrate is used, a labeled fragment having a size of approximately 30 bp or less is generated only when the incision by the XPG inevitably take place. Therefore, a level of the XPG activity may be quantitatively analyzed by determining an amount of the generated labeled fragment having a size of approximately 30 bp or less.

There is no limitation to cells or tissues which become a sample used to extract the biological extract. The cells or tissues may be used without limitation as long as they are required to analyze the XPG endonuclease activity. For example, the biological extract may include all eukaryotic cells or tissues of mammals including humans. According to one exemplary embodiment of the present invention for analyzing the XPG endonuclease activity, human fibroblasts or RKO cells or rat tissues may be used as a target, but the present invention is not limited thereto. The cells or tissues may be selected and used by those skilled in the art, depending on a purpose of analysis.

In this specification, the term “biological extract” means an extract including proteins extracted from the nuclei, cells and/or tissue of an organism. More particularly, the biological extract may include a cellular nuclear extract, a total cellular protein extract and/or a total tissue protein extract.

In this specification, the term “cellular nuclear extract” means contents including all proteins in the nuclei extracted after removal of protoplasm and nuclear membrane from the eukaryotic cells. In this case, the cellular nuclear extract is used to differentiate from a purified protein. According to one exemplary embodiment, the nuclear extract may be obtained by treating the cells with a phosphotase inhibitor and a protease inhibitor, but the present invention is not limited thereto. However, the nuclear extract may be extracted using any extraction method known in the art.

In this specification, the term “total cellular protein extract” means contents including all intracellular proteins extracted by lysing the eukaryotic cells. In this case, the total cellular protein extract is used to differentiate from a purified protein. According to one exemplary embodiment, the total cellular protein extract may be obtained by treating the cells with a protease inhibitor, but the present invention is not limited thereto. However, the total cellular protein extract may be extracted using any extraction method known in the art.

In this specification, the term “total tissue protein extract” means contents including all intracellular proteins extracted by lysing a certain tissue (for example, liver, kidney, lungs, stomach, etc.) of an organism. In this case, the total tissue protein extract is used to differentiate from a purified protein. According to one exemplary embodiment, the total tissue protein extract may be obtained by adding tissues to a lysis buffer and homogenizing the tissues, but the present invention is not limited thereto. However, the total tissue protein extract may be extracted using any extraction method known in the art.

In this specification, the term “DNA bubble substrate” means a DNA construct composed of DNA double strands whose termini complementarily bind to each other to form a double-stranded DNA but some of the double-strand DNA does not complementarily bind to each other in the middle, thereby forming a bubble-like shape. The bubble substrate acts as a substrate of XPG endonuclease since a bubble patch which does not form a DNA pair of two strands is recognized as a damaged patch. In this case, the XPG recognizes a bubble structure of the bubble substrate and incises the 3′ terminus of the bubble structure.

According to one exemplary embodiment of the present invention, the bubble substrate may have oligonucleotides set forth in SEQ ID NO: 1 (upper strand: 5′-CCA GTG ATC ACA TAC GCT TTG CTA GGA CAT CCC CCC CCC CCC CCC CCC CCC CCC CCC CCC CAG TGC CAC GTT GTA TGC CCA CGT TGA CCG-3′) and SEQ ID NO: 2 (down strand: 5′-CGG TCA ACG TGG GCA TAC AAC GTG GCA CTG TTT TTT TTT TTT TTT TTT TTT TTT TTT TTT ATG TCC TAG CAA AGC GTA TGT GAT CAC TGG-3′) which complementarily bind to each other. The oligonucleotides of SEQ ID NO: 1 and SEQ ID NO: 2 have a bubble shape in which both termini complementarily bind to each other to form double-stranded DNA, but do not bind to each other in patches of a C repeated sequence (SEQ ID NO: 1) and a T repeated sequence (SEQ ID NO: 2) in the middle, thereby forming a bubble shape.

According to another exemplary embodiment of the present invention, the analysis method according to the present invention is characterized in that it uses a DNA bubble substrate having a detectable label attached to the 3′-terminus thereof. In this case, the detectable label may be a radioactive isotope, a fluorescent material, a luminescent material or a precursor of the luminescent material. More particularly, the detectable label may be selected from the group consisting of ³²P, ³⁵S, ¹³¹I, ¹²³I, ¹²⁵I, ³H, carboxyfluorescein (FAM), tetramethylrhodamine (TAMRA), Cy3, Cy5, IRDye series, fluorescein, fluorescein isothiocyanate (FITC), rhodamine, Texas Red, Alexa series, digoxigenin (DIG) and biotin.

A method of attaching the detectable label to a DNA bubble substrate is widely known in the art. Thus, a person having skills in the art may properly select and use one of known methods.

According to sill another exemplary embodiment of the present invention, the mixing of the biological extract and the bubble substrate may be performed in the presence of a reaction buffer. In this case, the reaction buffer may be, for example, a HEPES buffer, but the present invention is not limited thereto

According to sill another exemplary embodiment of the present invention, the reaction buffer may have a pH value of 6.0 to 8.5. More particularly, the pH value may be in a range of pH 6.0 to 8.0, pH 6.5 to 8.0, pH 6.5 to 7.5, or pH 6.5 to 7.0. This pH range is a pH range in which the optimum binding of the XPG to the DNA bubble substrate may take place.

According to yet another exemplary embodiment of the present invention, the method according to the present invention may further include adding MgCl₂ or MnCl₂ as a co-factor in the mixing of the biological extract and the bubble substrate. In this case, the MgCl₂ or MnCl₂ may be in advance included in the reaction buffer. An amount of the added MgCl₂ or MnCl₂ may be in a range of 2 to 10 mM. This is a content range in which the optimum binding of the XPG to the DNA bubble substrate may take place.

The present inventors have conducted research to quantitatively analyze an endonuclease activity of XPG derived from an endogenous protein extract. The “DNA bubble” having a detectable label (for example, a radioactive isotope label or a non-radioactive material label) attached to the 3′-terminus thereof is an XPG-like substrate. Here, when the substrate encounters a biological extract including the XPG (i.e., a cellular nuclear protein extract, a total cellular protein extract and/or a total tissue protein extract), the DNA incision may successfully take place in a structure-specific manner. According to one exemplary embodiment of the present invention, nucleus protein extracts extracted from normal fibroblasts and human colon cancer RKO cells were compared with XPG-deficient cells (i.e., XPG-null fibroblasts and XPG knock-down RKO cells, respectively). In comparison with a purified human XPG protein, it was revealed that analysis of the XPG incision of a total tissue protein extract extracted from human and rat tissues (liver and kidney) was successfully established. In addition, the analysis was optimized on every sample type under different conditions. Accordingly, the direct analysis method based on endogenous XPG according to the present invention has main advantages in that it is not labor-intensive, economical, and highly reproducible. The XPG analysis according to the present invention is applicable to most cells and tissues of interest.

### BRIEF DESCRIPTION OF THE DRAWINGS

The above and other objects, features and advantages of the present invention will become more apparent to those of ordinary skill in the art by describing in detail exemplary embodiments thereof with reference to the attached drawings, in which:

FIG. 1 shows a method of analyzing an XPG activity using a bubble nucleotide substrate whose 3′- and 5′-termini are labeled and the analysis results. FIG. 1A schematically shows the analysis method using a bubble substrate whose 3′- and 5′-termini are labeled, showing that an asterisk (*) represents a radioactive label, and FIG. 1B shows the analysis results using a bubble substrate whose 3′- and 5′-termini are labeled.

FIG. 2 shows the results obtained by testing the XPG incision activities in the intracellular nuclei in a time-dependent (A) or dose-dependent (B) manner.

FIG. 3 shows the effects of main factors on the XPG activity. FIGS. 3A, 3B and 3C show the results according to changes in MgCl₂, MnCl₂, and buffer pH, respectively.

FIG. 4 shows the analysis results of the radioactivity-based quantitative XPG activity in various cell lines. FIG. 4A shows the results obtained by western blotting relative amounts of XPG expressed from nuclear extracts of normal XPG cells and XPG-deficient cells. FIG. 4B shows the results obtained by analyzing the XPG incision activity using the nuclear extracts of the normal XPG cells and the XPG-deficient cells. A purified human XPG protein, (+)hXPG, was used as a positive control.

FIG. 5 shows the results obtained by analyzing the in vitro and in vivo radioactivity-based XPG incision activities. FIG. 5A shows the results obtained by analyzing the XPG incision activity using a total cellular protein extract from normal XPG cells (RKO). FIG. 5B shows the results obtained by analyzing the XPG incision activity using a total tissue protein extract from rat tissues (liver).

FIG. 6 shows the results obtained by analyzing the in vitro non-radioactivity-based XPG incision activities. FIG. 6A shows the results obtained by comparing the intensities of spots, which were obtained by serially diluting a sample (lower panel) having a 0 to 100% biotin range and a biotin oligo standard (upper panel). FIG. 6B shows the results obtained by analyzing the XPG incision activity using a nucleus protein extract from the normal XPG cells (RKO). FIG. 6C shows the results obtained by analyzing the XPG incision activity using a total cellular protein extract from the normal XPG cells (RKO).

### DETAILED DESCRIPTION OF EXEMPLARY EMBODIMENTS

Hereinafter, exemplary embodiments of the present invention will be described in detail. However, the present invention is not limited to the embodiments disclosed below, but can be implemented in various forms. The following embodiments are described in order to enable those of ordinary skill in the art to embody and practice the present invention.

The terminology used herein is for the purpose of describing particular embodiments only and is not intended to be limiting of exemplary embodiments. The singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements, components and/or groups thereof, but do not preclude the presence or addition of one or more other features, integers, steps, operations, elements, components and/or groups thereof.

With reference to the appended drawings, exemplary embodiments of the present invention will be described in detail below. To aid in understanding the present invention, like numbers refer to like elements throughout the description of the figures, and the description of the same elements will be not reiterated.

### EXAMPLE 1

### Materials and Methods

1-1: Cell Culture

XPG shRNA (short-hairpin RNA) knock-down cells constructed using human colon cancer RKO cells (ATCC NO. CRL-257) were cultured in an RPMI 1640 medium supplemented with 10% fetal bovine serum (FBS) and 0.5 mg/ml puromycin, and wild-type RKO cells were cultured in an RPMI 1640 medium supplemented with 10% FBS (ATCC NO. CRL-257). Normal human fibroblast (GM08399) and XPG-null fibroblast (GM16398) cells (Coriell Cell Repository, Camden, N.J., USA) were cultured in a DMEM medium supplemented with 10% FBS. The culturing was performed at 37° C. and 5% CO₂.

The knock-down of XPG using a shRNA vector was performed, as follows: A plasmid was extracted (plasmid extraction) from an Escherichia coli clone, which included a retroviral vector pSM2c (Open Biosystems, USA) containing an XPG-specific shRNA target, using a HiSpeed Plasmid Midi kit (Qiagen, Germany). To confirm the presence of the XPG-specific shRNA target, the purified plasmid was sequenced (Sense: CCCACAGACTCAGTTCCAA, and Antisense: TTGGAACTGAGTCTGTGGG). The XPG shRNA knock-down cells were prepared using a transfecting reagent Fugen6 (Roche, Germany).

1-2: Animal Care

5-Week-old Sprague-Dawley rats with a body weight of approximately 200 g were purchased from Orient Bio Inc. (Seongnam, Korea). These animals were housed in a plastic cage in a laboratory which was well ventilated. A laboratory temperature and a relative humidity were maintained at 20±2° C. and 60±10%, respectively, with a day/night cycle of 12 hours. The rats were fed ad libitum with water and laboratory complete food. These animals were adapted 7 days before experiments. All the animal experiments were carried out according to the Animal and Ethics Review Committee in the Sookmyung Women's University.

1-3: Preparation of Cellular Nuclear Extract

A nuclear extract was prepared using a Caymans Nuclear Extraction kit (Caymanchem, USA). More particularly, 1×10⁶ RKO cells and fibroblasts were seeded in a 100-mm petri dish. After centrifugation, the cell pellets were washed twice with 5 ml of ice-cold PBS in the presence of a phosphatase inhibitor, and centrifuged again. The pellets were re-suspended in 500 μl of a cold (1×) hypotonic buffer, and incubated in ice for 15 minutes. 50 μl of 10% Nonidet P-40 was added to lyse a cell membrane, and then simply centrifuged at 14,000 g for 30 seconds. The pelleted nuclei were lysed in 50 μl of a cold (1×) complete nuclear extraction buffer containing a mixture of protease and a phosphotase inhibitor, and then vortexed. After the centrifugation, a cellular nuclear supernatant was collected, and stored at −80° C. Thereafter, the protein concentrate was quantitatively analyzed.

1-4: Preparation of Total Cellular Protein Extract

1×10⁶ Cells in 10 ml of a suspension were seeded and cultured in a 100-mm petri dish. Then, the cells were collected, and centrifuged at 1,500 rpm for 5 minutes to precipitate the cells. Thereafter, the cells were lysed for 30 minutes in 100 μl of an RIPA lysis buffer (50 mM Tris-HCl (pH 7.5), 0.5 mM ethylenediaminetetraacetic acid (EDTA), 150 mM NaCl, 1% (v/v) Triton-100, 0.1% (v/v) sodium dodecyl sulfate (SDS), 1 mM dithiothreitol (DTT) and a protease inhibitor cocktail) on an ice both. The entire cell lysate was centrifuged at 13,000 rpm for 30 minutes, and then stored at −80° C. The protein was quantitatively analyzed.

1-5: Preparation of Total Tissue Protein Extract

10 to 40 mg of liver and kidney tissues of a rat was added to 500 μl of a lysis buffer PRO-PREP (Intron Biotechnology, Republic of Korea), and minced with surgical forceps in an ice both. The minced tissues were homogenized, vortexed, and incubated for 30 minutes in ice. A supernatant was collected, and centrifuged at 13,000 rpm for 15 minutes, and the sample was divided, and stored −80° C. Finally, the protein concentrate were quantitatively analyzed.

1-6: Confirmation of Cellular Nuclear XPG Using Western Blotting Assay

A nuclear extract was separated through 10% SDS-PAGE, transferred to a polyvinylidene difluoride (PVDF) membrane, and then blocked. A mouse monoclonal antibody against XPG and peroxidase-conjugated goat anti-mouse IgG (Santa Cruz Biotechnology, USA) were used as secondary antibodies to detect an XPG protein. Thereafter, a chemiluminescent method was performed (ECL Plus Western blotting detection kit, GE Healthcare, UK). CyclinB1 was used as a protein loading control. The whole procedures were carried out in triplicate.

1-7: Analysis Method Based on a Radiometric System

Preparation of DNA Bubble Substrate whose 3′ Terminus is Labeled with Radioactive Isotope

A bubble oligonucleotide substrate was used as a specific substrate to analyze the XPG incision activity. First, the 3′-terminus of a single strand was labeled with terminal transferase (NEB, England) and [α-³²P]dATP, as follows. As the first DNA strand, 20 pmol of bubble-up oligonucleotide (SEQ ID NO: 1) was added to a labeled reaction mixture (50 μl) including a (1×) terminal transferase buffer, 250 mM CoCl₂, 100 mM [α-³²P]dATP and 10 units of terminal transferase. The resulting mixture was incubated at 37° C. for 30 minutes, and then inactivated at 70° C. for 10 minutes. The finally labeled bubble-up oligonucleotide was precipitated from 1 μl of glycogen and 36 μl of isopropanol, and centrifuged at 15,000 rpm for 10 minutes at room temperature. Then, an annealing step was performed by adding 38 μl of a (1×) annealing buffer (10 mM Tris pH 8.0, 1 mM EDTA and 100 mM NaCl) and an equivalent amount of the second non-labeled DNA strand, bubble-down oligonucleotide (SEQ ID NO: 2), and cooling the resulting mixture from 95° C. in a water both.

Preparation of DNA Bubble Substrate whose 5′ Terminus is Labeled with Radioactive Isotope

A procedure of labeling a bubble substrate with a 5′-terminus radioactive isotope was performed in substantially the same manner as in the procedure of labeling the bubble substrate with the 3′-terminus radioactive isotope as described above in Example 1-6. The first bubble-down oligonucleotide strand was first 5′-labeled with T4 polynucleotide kinase (Promega, USA) and [γ-³²P]ATP. Thereafter, 20 pmol of the second bubble-up oligonucleotide was added to a labeled reaction mixture (50 μl) including a (1×) T4 polynucleotide kinase buffer, [γ-³²P]ATP and 20 units of T4 polynucleotide kinase. The subsequent procedures were performed in the same manner as in the procedure of labeling the bubble substrate with the 3′-terminus radioactive isotope.

Preparation of DNA Marker Labeled with Radioactive Isotope

The 5′-terminus of a DNA marker was labeled with a radioactive isotope using [γ-³²P]ATP and T4 polynucleotide kinase. The DNA size marker used herein was dephosphorylated Φx174 DNA/HinfI (Promega,USA). Basically, 50 to 200 ng of the DNA marker was mixed with 10 μl of a reaction mixture ((1×) T4 polynucleotide kinase buffer, 2 mM └γ-³²P┘ATP and 10 units of T4 polynucleotide kinase), and then cultured at 37° C. for 10 minutes. An equivalent volume of a (1×) sample loading buffer (90% formamide, 5 mM EDTA, 0.1% bromophenol blue and 0.1% xylene cyanol) was added to stop the reaction. Before gel electrophoresis, the sample was heated at 95° C. for 3 minutes.

Radioactivity-Based XPG Incision Assay Using Cellular Nuclear Extract

An XPG endonuclease assay was performed using a slight modification based on the in vitro analysis procedure as described in E. Evans et al. (E. Evans, J.

Fellow, A. Coffer, and R. D. Wood, EMBO. 16 (1997) 625-638) and Y. Habraken et al. (A. Constantinou, D. Gunz, E. Evans, P. Lalle, P. A. Bates, R. D. Wood, and S. G. Glarkson, J. Biol. Chem. 274 (1999) 5637-5648). A nuclear extract (generally used in a range of 50 to100 ng) and 1 pmol of a radioactive isotope-labeled bubble oligonucleotide substrate were mixed with 8 μl of a reaction buffer (25 mM HEPES (pH 6.8), 10% glycerol, 2 mM MgCl₂, 50 mg/ml of bovine serum albumin (BSA) and 1 mM DTT) at 37° C. for 30 minutes. The reaction was stopped by adding an equivalent volume of a (1×) sample loading buffer. The sample was heated at 95° C. for 3 minutes, and then separated through 12% SDS-PAGE (at 1,000 V for 1 hour). Thereafter, the gel was transferred to a Whatman® 3-mm paper, dried and visualized using autoradiography. All the assays were performed in duplicate.

Radioactivity-Based XPG Incision Assay Using Total Cellular Protein Extract

An XPG endonuclease assay was performed using a slight modification based on the in vitro analysis procedure as described in E. Evans et al. (E. Evans, J. Fellow, A. Coffer, and R. D. Wood, EMBO. 16 (1997) 625-638) and Y. Habraken et al. (A. Constantinou, D. Gunz, E. Evans, P. Lalle, P. A. Bates, R. D. Wood, and S. G. Glarkson, J. Biol. Chem. 274 (1999) 5637-5648). A total cellular protein extract (generally 50 to 100 ng of a protein used) and 1 pmol of a radioactive isotope-labeled bubble oligonucleotide substrate were mixed with 8 μl of a reaction buffer (25 mM HEPES (pH 6.8), 10% glycerol, 2 mM MgCl₂, 50 mg/ml of bovine serum albumin (BSA) and 1 mM DTT) at 37° C. for 30 minutes. The reaction was stopped by adding an equivalent volume of a (1×) sample loading buffer. The sample was heated at 95° C. for 3 minutes, and then separated through 12% SDS-PAGE (at 1,000 V for 1 hour). Thereafter, the gel was transferred to a Whatman® 3-mm paper, dried and visualized using autoradiography. All the assays were performed in duplicate.

Radioactivity-Based XPG Incision Assay Using Total Tissue Protein Extract

An XPG endonuclease assay was performed using a slight modification based on the in vitro analysis procedure as described in E. Evans et al. (E. Evans, J. Fellow, A. Coffer, and R. D. Wood, EMBO. 16 (1997) 625-638) and Y. Habraken et al. (A. Constantinou, D. Gunz, E. Evans, P. Lalle, P. A. Bates, R. D. Wood, and S. G. Glarkson, J. Biol. Chem. 274 (1999) 5637-5648). A total tissue protein extract (generally 500 to 1,000 ng of a protein used) and 1 pmol of a radioactive isotope-labeled bubble oligonucleotide substrate were mixed with 8 μl of a reaction buffer (25 mM HEPES (pH 6.8), 10% glycerol, 2 mM MgCl₂, 50 mg/ml of bovine serum albumin (BSA) and 1 mM DTT) at 37° C. for 30 minutes. The reaction was stopped by adding an equivalent volume of a (1×) sample loading buffer. The sample was heated at 95° C. for 3 minutes, and then separated through 12% SDS-PAGE (at 1,000 V for 1 hour). Thereafter, the gel was transferred to a Whatman® 3-mm paper, dried and visualized using autoradiography. All the assays were performed in duplicate.

1-8: Analysis Method Based on Non-Radiometric System

Preparation of DNA Bubble Substrate whose 3′-Terminus is Labeled with Biotin

Example 1-7 disclosed the method when the radioactive isotope was used as the detectable label bound to the 3′-terminus of the DNA bubble. In this Example, a method using a label other than the radioactive isotope was described. A bubble oligonucleotide substrate was used as a specific substrate to analyze the XPG incision activity. First, the 3′-terminus of a single strand was labeled with terminal transferase (Thermo Scientific, USA) and biotin-11-UTP to prepare the biotinylated 3′-terminus. As the first DNA strand, 5 pmol of bubble-up oligonucleotide (SEQ ID NO: 1) was added to a labeled reaction mixture (50 μl) including a (1×) terminal transferase buffer, 0.5 nM biotin-11-UTP, and 1 unit of terminal transferase (Thermo Scientific, USA). The resulting mixture was incubated at 37° C. for 30 minutes. The finally labeled bubble-up oligonucleotide was precipitated from 1 μl of glycogen and 36 μl of isopropanol, and then centrifuged at 15,000 rpm for 10 minutes at room temperature. Then, an annealing step was performed by adding 38 μl of a (1×) annealing buffer (10 mM Tris pH 8.0, 1 mM EDTA and 100 mM NaCl) and an equivalent amount of the second non-labeled DNA strand, bubble-down oligonucleotide (SEQ ID NO: 2), and cooling the resulting mixture from 60° C. in a water both. The sample was stored at −20° C. until use.

Preparation of Biotin-Labeled DNA Marker

Biotin-11-UTP and T4 polynucleotide kinase were used to label the 5′-terminus of a DNA marker with biotin. The DNA size marker used herein was dephosphorylated Φx174 DNA/HinfI (Promega,USA). Basically, 50 to 200 ng of the DNA marker was mixed with 10 ml of a reaction mixture ((1×) T4 polynucleotide kinase buffer, 0.5 nM biotin-11-UTP and 10 units of T4 polynucleotide kinase), and then cultured at 37° C. for 10 minutes. An equivalent volume of a (1×) sample loading buffer (90% formamide, 5 mM EDTA, 0.1% bromophenol blue and 0.1% xylene cyanol) was added to stop the reaction. Before gel electrophoresis, the sample was heated at 95° C. for 3 minutes. The sample was stored at −20° C. until use.

Non-Radioactivity-Based XPG Incision Assay Using Cellular Nuclear Extract

An XPG endonuclease assay was performed using a slight modification based on the in vitro analysis procedure as described in E. Evans et al. (E. Evans, J. Fellow, A. Coffer, and R. D. Wood, EMBO. 16 (1997) 625-638) and Y. Habraken et al. (A. Constantinou, D. Gunz, E. Evans, P. Lalle, P. A. Bates, R. D. Wood, and S. G. Glarkson, J. Biol. Chem. 274 (1999) 5637-5648). A nuclear extract (generally used in a range of 50 to 100 ng) and 1 pmol of a bubble oligonucleotide substrate whose 3′-terminus was labeled with biotin were mixed with 8 μl of a reaction buffer (25 mM HEPES (pH 6.8), 10% glycerol, 2 mM MgCl₂, 50 mg/ml of bovine serum albumin (BSA) and 1 mM DTT) at 37° C. for 30 minutes. The reaction was stopped by adding an equivalent volume of a (1×) sample loading buffer. The sample was separated through 12% SDS-PAGE (at 100 V for 3 hour). Thereafter, the gel was transferred to a positively charged nylon membrane through electrophoresis at 25 V/250 mA for 1 hour, and immobilized through UVC cross-linking at 0.120 J/cm² for 1 minute. The membrane was sensed by a chemiluminescent signal using a Chemilunescent Nucleic Acid Detection module (Thermo Scientific, USA). Briefly, the biotinylated DNA cleavage banding patterns by the XPG endonuclease activity were chemiluminescent materially detected using Chemiluminescent material Nucleic Acid Detection Module (Thermo Scientific, USA) in according to the manufacturer's instructions. In brief, the blotted membrane was initially blocked with a pre-warmed blocking buffer (37 to 50° C.) for 15 minutes with gentle shaking, and then incubated with a conjugate/blocking buffer solution containing a stabilized streptavidin-horseradish peroxidase conjugate (1:300 dilution) for 15 minutes with gentle shaking. The membrane was rinsed briefly and washed four times with a 1× wash buffer for 5 minutes each. Prior to signal enhancement, the membrane was equilibrated with a substrate equilibration buffer for 5 minutes with gentle shaking. Subsequently, the membrane was carefully removed from the substrate equilibration buffer and then placed in a clean container containing a substrate working solution (a luminol/enhancer solution: a stable peroxide solution as mixture ratio of 1:1) for 5 minutes without shaking. Finally, the membrane was removed from the substrate working solution by blotting an edge of the membrane on a paper towel to remove excess buffer, exposed to an X-ray film for 2 to 5 minutes (depending on the desired signal), and subjected to film development according to the manufacturer's recommendations. All the assays were performed independently in duplicate.

Non-Radioactivity-Based XPG Incision Assay Using Total Cellular Protein Extract

An XPG endonuclease assay was performed using a slight modification based on the in vitro analysis procedure as described in E. Evans et al. (E. Evans, J. Fellow, A. Coffer, and R. D. Wood, EMBO. 16 (1997) 625-638) and Y. Habraken et al. (A. Constantinou, D. Gunz, E. Evans, P. Lalle, P. A. Bates, R. D. Wood, and S. G. Glarkson, J. Biol. Chem. 274 (1999) 5637-5648). A total cellular protein extract (generally 50 to 100 ng of a protein used) and 1 pmol of a bubble oligonucleotide substrate whose 3′-terminus was labeled with biotin were mixed with 8 μl of a reaction buffer (25 mM HEPES (pH 6.8), 10% glycerol, 2 mM MgCl₂, 50 mg/ml of bovine serum albumin (BSA) and 1 mM DTT) at 37° C. for 30 minutes. The reaction was stopped by adding an equivalent volume of a (1×) sample loading buffer. The sample was separated through 12% SDS-PAGE (at 100 V for 3 hour). Thereafter, the gel was transferred to a positively charged nylon membrane through electrophoresis at 25 V/250 mA for 1 hour, and immobilized through UVC cross-linking at 0.120 J/cm² for 1 minute. The membrane was sensed by a chemiluminescent signal using a Chemilunescent Nucleic Acid Detection module (Thermo Scientific, USA). All the assays were performed in duplicate.

Non-Radioactivity-Based XPG Incision Assay Using Total Tissue Protein Extract

An XPG endonuclease assay was performed using a slight modification based on the in vitro analysis procedure as described in E. Evans et al. (E. Evans, J. Fellow, A. Coffer, and R. D. Wood, EMBO. 16 (1997) 625-638) and Y. Habraken et al. (A. Constantinou, D. Gunz, E. Evans, P. Lalle, P. A. Bates, R. D. Wood, and S. G. Glarkson, J. Biol. Chem. 274 (1999) 5637-5648). A total tissue protein extract (generally 500 to 1,000 ng of a protein used) and 1 pmol of a bubble oligonucleotide substrate whose 3′-terminus was labeled with biotin were mixed with 8 μl of a reaction buffer (25 mM HEPES (pH 6.8), 10% glycerol, 2 mM MgCl₂, 50 mg/ml of bovine serum albumin (BSA) and 1 mM DTT) at 37° C. for 30 minutes. The reaction was stopped by adding an equivalent volume of a (1×) sample loading buffer. The sample was separated through 12% SDS-PAGE (at 100 V for 3 hour). Thereafter, the gel was transferred to a positively charged nylon membrane through electrophoresis at 25 V/250 mA for 1 hour, and immobilized through UVC cross-linking at 0.120 J/cm² for 1 minute. The membrane was sensed by a chemiluminescent signal using a Chemilunescent Nucleic Acid Detection module (Thermo Scientific, USA). All the assays were performed in duplicate.

### EXAMPLE 2

### XPG Activity Assay

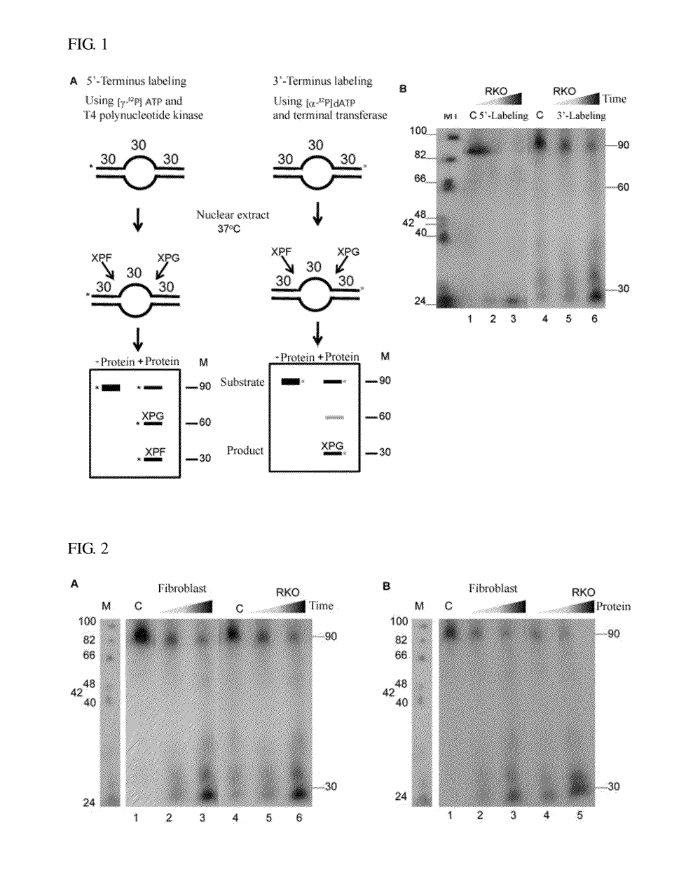

In the prior art, XPG was purified and used in the XPG incision activity assay. In particular, since an ERCC1-XPF complex incised the 5′-terminus of a substrate, the typical XPG activity assay was not satisfactorily performed (FIG. 1A). To solve these problems, the present invention provides a method of analyzing an XPG incision activity by labeling the 3′-terminus of the substrate with a detectable label (FIG. 1B).

In FIG. 1B, the left panel shows the results using a 5′-terminus-labeled bubble substrate, and the right panel shows the results using a 3′-terminus-labeled bubble substrate. C represents a negative control which did not include a nuclear extract, M represents a DNA marker, Lanes 2 and 3 represent the results obtained by reaction for 10 and 30 minutes, respectively, using the 5′-terminus-labeled substrate, and Lanes 5 and 6 represent the results obtained by reaction for 10 and 30 minutes, respectively, using the 3′-terminus-labeled substrate.

As shown in FIG. 1B, it was confirmed that, when the 5′-terminus labeled substrate is used, a nucleotide fragment having a size of approximately 60 bp, which could confirm the presence of the XPG incision activity, was not observed, whereas nucleotide fragments having a size of less than 30 bp were generated due to an effect of the ERCC1-XPF complex having the 5′-terminus incision activity (Lanes 1 to 3).

However, it was confirmed that, when the 3′-terminus labeled substrate was used, a larger amount of the nucleotide fragment having a size of approximately 30 bp, which could confirm the presence of the XPG incision activity in the presence of the nuclear extract (Lanes 5 and 6) compared with the absence of the nuclear extract (Lane 4).

Form these results, it could be seen that the 3′-terminus-labeled bubble substrate was a suitable substrate to effectively analyze the XPG activity.

### EXAMPLE 3

### Time- or Dose-Dependent XPG Activity Assay

Based on the results as described above, the XPG incision activity with respect to the bubble substrate whose 3′-terminus was labeled with a radioactive isotope was analyzed in a time- or dose-dependent manner. Unless stated explicitly otherwise, the mixture of the 3′-terminus-labeled bubble substrate and the nuclear extract was cultured under the standard XPG assay conditions in a time-dependent manner. The XPG in the nuclear extract may specifically incise the substrate. In FIG. 2A, C represents a control which did not include a nuclear extract, M represents a DNA marker, Lane 2 or 5 represents the results obtained by incubating a substrate for 10 minutes, and Lane 3 or 6 represents the results obtained by incubating a substrate for 30 minutes. When the XPG nuclear extract was present, the incised products showed constant mobility, which corresponds to a size of approximately 30 bp. An amount of the nucleotide-incised product was increased with an increase in culture time, which was in inverse proportion to an amount of the unincised substrate. From these results, it could be seen that the incision of the DNA bubble substrate with the XPG nuclear extract was performed in a time-dependent manner. As another experiment, the dependence of the XPG endonuclease activity on a concentration of an extract was tested. In FIG. 2B, C represents a negative control which did not include a nuclear extract, M represents a DNA marker, Lane 2 or 4 represents the results obtained by adding 50 ng of a nuclear extract, and Lane 3 or 5 represents the results obtained by adding 100 ng of a nuclear extract. From the experiment results, it was confirmed that the XPG incision activity was in proportion to an amount of the nuclear extract.

As a result, it could be seen that the analysis of the XPG endonuclease activity using the nuclear extract were observed in a time- and dose-dependent manner.

### EXAMPLE 4

### Evaluation of Effects of Factors on XPG Activity

Effects of different parameters (pH of a divalent co-factor and a reaction buffer) on XPG incision were tested. An XPG incision reaction was reconstructed in the presence of various discrete factors so that 100 pmol of a bubble substrate whose 3′-terminus was labeled with a radioactive isotope and 100 ng of a nuclear extract can be cultured at 37° C. for 30 minutes in a reaction buffer, and the DNA banding patterns and affinities were then measured.

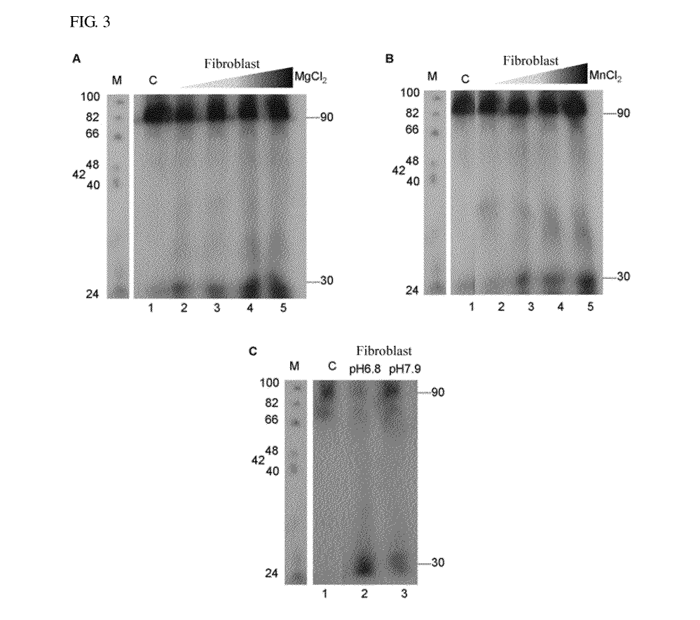

First, the bubble substrate whose 3′-terminus was labeled with a radioactive isotope and the nuclear extract were mixed in the presence of MgCl₂ or MnCl₂ (2 to 10 mM), and then incubated. FIG. 3A shows the results on an effect of MgCl₂. Here, C represents a negative control, M represents a DNA marker, and Lanes 2, 3, 4 and 5 represent the results obtained by adding 2 mM, 5 mM, 7 mM and 10 mM of MgCl₂, respectively. An increase in concentration of MgCl₂ showed improved XPG reaction efficiency, and the most effective optimum XPG reaction was observed when MgCl₂ was used at a concentration of 7 mM. FIG. 3B shows the results on an effect of MnCl₂. Here, C represents a negative control, M represents a DNA marker, and Lanes 2, 3, 4 and 5 represent the results obtained by adding 2 mM, 5 mM, 7 mM and 10 mM of MnCl₂. Similarly, the improved XPG endonuclease activity was observed with an increase in concentration of MnCl₂.

To confirm an effect of pH of a reaction buffer on the XPG incision efficiency of the nuclear extract, experiments were performed using a HEPES buffer with pH 6.8 or pH 7.9. In FIG. 3C, C represents a negative control, and M represents a DNA marker. Here, the XPG endonuclease activity was observed in both of the reaction buffers with pH 7.9 and pH 6.8, but a higher level of the XPG endonuclease activity was observed in the reaction buffer with pH 6.8, compared with the reaction buffer with pH 7.9.

### EXAMPLE 5

### Comparison of Radioactivity-Based XPG Activity Between XPG Normal Cells and XPG-Deficiency Cells

Quantitative analysis of the XPG endonuclease activities in human fibroblasts and human colon cancer RKO cells was performed to compare with the XPG-deficient cells.

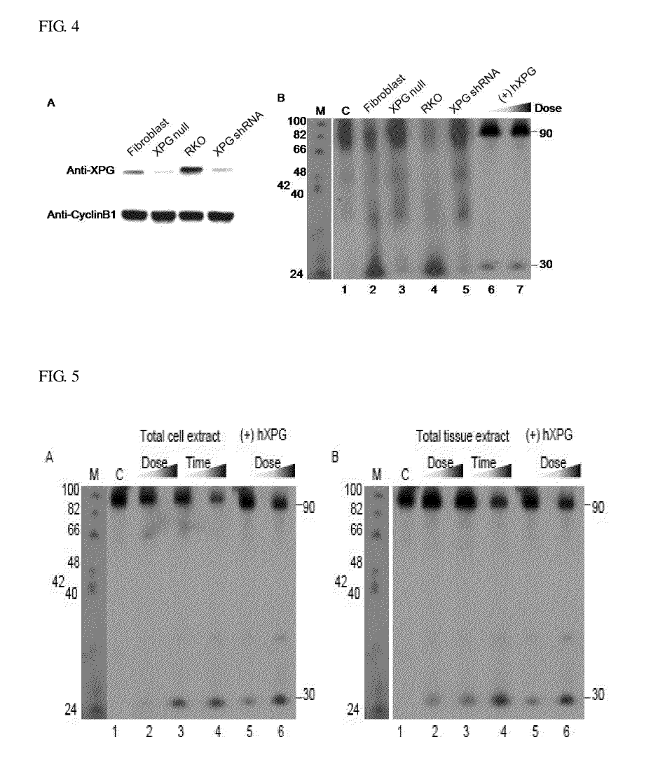

FIG. 4A shows the results obtained by western-blotting relative amounts of XPG proteins in the nuclear extracts extracted from normal XPG cells (fibroblasts and RKO) and XPG-deficient cells (XPG-null fibroblasts and XPG shRNA knock-down RKO cells). CyclinB1 represents a loading control. It was confirmed that an expression level of XPG was significantly reduced in the XPG-deficient cell-derived nuclear extract, compared with the normal cell-derived nuclear extract.

FIG. 4B shows the results obtained by incubating various cell-derived XPG nuclear extracts and a bubble substrate whose 3′ terminus was labeled with a radioactive isotope in a reaction buffer (pH 6.8) including 7 mM MgCl₂. Here, C represents a negative control, M represents a DNA marker, and (+)hXPG represents a purified human XPG protein as a positive control. It was confirmed that a relatively high level of the XPG-mediated DNA incision took place in the normal fibroblasts (Lane 2), compared with the XPG-null cells (Lane 3). Similarly, a relatively high level of the DNA incision activity of XPG was also observed in the RKO cells (Lane 4), compared with the XPG shRNA knock-down cells (Lane 5). It could be seen that the DNA incision activity of the XPG nuclear extract according to the present invention was highly reproducible, compared with the purified recombinant human XPG activities (FIG. 4B, Lanes 6 and 7) that was a positive control. From such results, the optimized analysis conditions for the XPG activity can be confirmed according to the present invention.

### EXAMPLE 6

### In Vitro and In Vivo Radioactivity-Based XPG Activities

After the XPG activity assay in the nuclear extract, an XPG endonuclease activity assay was carried out using the total protein extract from human cells and rat tissues. A purified human XPG protein was used as a positive control.

FIG. 5A shows the results obtained by analyzing the XPG incision activity using a total cellular protein extract from normal XPG cells (RKO). As the positive control, a purified human XPG protein, (+)hXPG, was considered to be a comparison target. The XPG activity against the bubble substrate whose 3′-terminus was labeled with a radioactive isotope (1 pmol) varied according to an amount of the total cellular protein extract and an incubation time. In the reaction buffer of 37° C., Lane 2 represents the results obtained using 50 ng of a substrate, Lanes 3 and 4 represent the results obtained using 100 ng of a substrate, Lane 5 represents the results obtained using 50 ng of a substrate, and Lane 6 represents the results obtained using 100 ng of a substrate. Also, Lanes 2 and 3 represent the results obtained by incubating a substrate for 10 minutes, Lane 4 represents the results obtained by incubating a substrate for 30 minutes, and Lanes 5 and 6 represent the results obtained by incubating a substrate for 30 minutes. C represents a negative control, and M represents a DNA marker. FIG. 5B shows the results obtained by analyzing the XPG incision activity using a total tissue protein extract from a rat tissue (liver). As the positive control, a purified human XPG protein, (+)hXPG, was considered to be a comparison target. The XPG activity against the bubble substrate whose 3′-terminus was labeled with a radioactive isotope (1 pmol) varied according to an amount of the total cellular protein extract and an incubation time. In the reaction buffer of 37° C., Lane 2 represents the results obtained using 500 ng of a substrate, Lanes 3 and 4 represent the results obtained using 1,000 ng of a substrate, Lane 5 represents the results obtained using 50 ng of a substrate, and Lane 6 represents the results obtained using 100 ng of a substrate. Also, Lanes 2 and 3 represent the results obtained by incubating a substrate for 10 minutes, Lane 4 represents the results obtained by incubating a substrate for 30 minutes, and Lanes 5 and 6 represent the results obtained by incubating a substrate for 30 minutes. C represents a negative control, and M represents a DNA marker.

When the total XPG extract (50 to 100 ng of a total cellular protein extract) derived from human cells was incubated for 10 to 30 minutes in a reaction buffer (pH 6.8) containing 7 mM MgCl₂, an incised product having approximately 30 nucleotides was generated. As shown in Lanes 2 and 3 and Lanes 3 and 4 of FIG. 5A, the incised product was generated in a time- and dose-dependent manner. Similar to these results, when the total XPG extract (500 to 1,000 ng of a total tissue protein extract) derived from rat tissues was incubated for 10 to 30 minutes in a reaction buffer (pH 6.8) containing 7 mM MgCl₂, an incised product having approximately 30 nucleotides was generated. As shown in Lanes 2 and 3 and Lanes 3 and 4 of FIG. 5B, the incised product was generated in a time- and dose-dependent manner. DNA patterns formed from the total protein extracts of the human cells or tissues were matched with a DNA pattern of the human XPG protein (Lanes 5 and 6 of FIGS. 5A and 5B) in the case of the positive control, which indicates the incised product was generated by the XPG endonuclease activity. These results shows that the method of analyzing an XPG activity using the total protein extract according to the present invention was successfully performed in vitro and in vivo.

### EXAMPLE 7

### In Vitro Non-Radioactivity-Based XPG Activities

A radioactive isotope was very expensive, hazardous and had a short life span. Therefore, as an alternative to the radioactive isotope, the 3′-terminus of a DNA bubble substrate was labeled with biotin. The 3′-terminus of the bubble substrate was labeled with biotin using the method as described in Example 1-8. 1-3 Biotinylated ribonucleotide was bound to the the 3′-terminus of a bubble-shaped oligonucleotide using terminal deoxynucleotidyl transferase in the presence of Co²⁺. Thereafter, biotin-mediated tailing of the bubble oligonucleotide was carried out. The labeling efficiency was determined using dot blotting, and the spot intensity of a spot sample was determined by a chemiluminescent method using a streptavidin-horseradish peroxidase (HRP) conjugate detection system, and compared with the biotin control oligo standard (0 to 100% biotin).

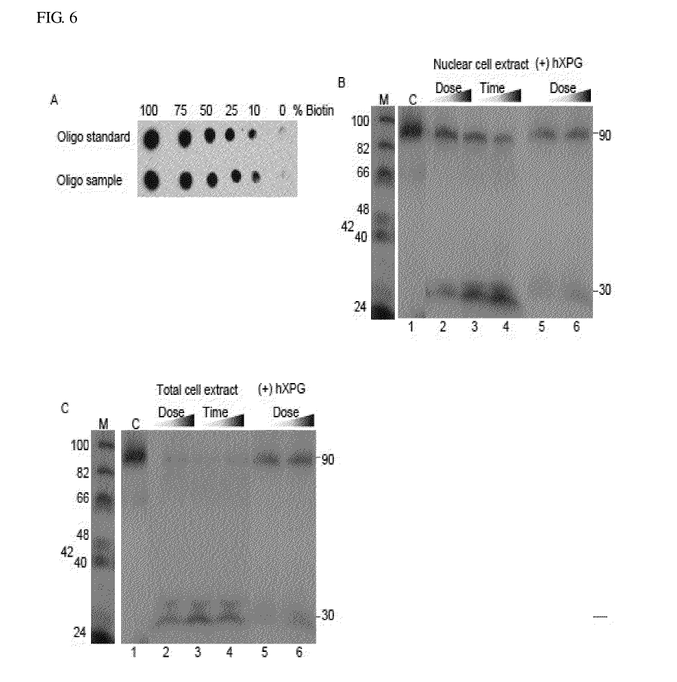

FIG. 6A shows the results obtained by comparing the intensities of spots, which were obtained by serially diluting a sample (lower panel) having a 0 to 100% biotin range and a biotin oligo standard (upper panel). As shown in the upper and lower panels of FIG. 6A, the biotin labeling efficiency of the sample might be compared with the control standard. FIG. 6B shows the results obtained by analyzing the XPG incision activity using a nucleus protein extract from the normal XPG cells (fibroblasts and RKO). As the positive control, a purified human XPG protein, (+)hXPG, was considered to be a comparison target. The XPG activity against the bubble substrate whose 3′-terminus was labeled with biotin (1 pmol) varied according to an amount of the nuclear extract and an incubation time. In the reaction buffer of 37° C., Lane 2 represents the results obtained using 50 ng of a substrate, Lanes 3 and 4 represent the results obtained using 100 ng of a substrate, Lane 5 represents the results obtained using 50 ng of a substrate, and Lane 6 represents the results obtained using 100 ng of a substrate. Also, Lanes 2 and 3 represent the results obtained by incubating a substrate for 10 minutes, Lane 4 represents the results obtained by incubating a substrate for 30 minutes, and Lanes 5 and 6 represent the results obtained by incubating a substrate for 30 minutes. C represents a negative control, and M represents a DNA marker. FIG. 6C shows the results obtained by analyzing the XPG incision activity using the total cellular protein extract from normal XPG cells (fibroblasts and RKO). As the positive control, a purified human XPG protein, (+)hXPG, was considered to be a comparison target. The XPG activity against the bubble substrate whose 3′-terminus was labeled with biotin (1 pmol) varied according to an amount of the nuclear extract and an incubation time. In the reaction buffer of 37° C., Lane 2 represents the results obtained using 50 ng of a substrate, Lanes 3 and 4 represent the results obtained using 100 ng of a substrate, Lane 5 represents the results obtained using 50 ng of a substrate, and Lane 6 represents the results obtained using 100 ng of a substrate. Also, Lanes 2 and 3 represent the results obtained by incubating a substrate for 10 minutes, Lane 4 represents the results obtained by incubating a substrate for 30 minutes, and Lanes 5 and 6 represent the results obtained by incubating a substrate for 30 minutes. C represents a negative control, and M represents a DNA marker.

When the nuclear extract (50 to 100 ng of a protein extract) from human cells was incubated for 10 to 30 minutes in a reaction buffer (pH 6.8) containing 7 mM MgCl₂, an incised product having approximately 30 nucleotides was generated. As shown in Lanes 2 and 3 and Lanes 3 and 4 of FIG. 6B, the incised product was generated in a time- and dose-dependent manner, and corresponded to the positive XPG-incised product as shown in Lanes 5 and 6 of FIG. 6B. Similar to these results, when the cells extract (50 to 100 ng of a total cellular protein extract) was incubated for 10 to 30 minutes in a reaction buffer (pH 6.8) containing 7 mM MgCl₂, an incised product having a size of approximately 30 nucleotides was generated. As shown in Lanes 2 and 3 and Lanes 3 and 4 of FIG. 6A, the incised product was generated in a time- and dose-dependent manner, and corresponded to the positive XPG-incised product as shown in Lanes 5 and 6 of FIG. 6C.

This indicates that the incised product of the DNA bubble substrate was generated by the XPG endonuclease activity. Also, it could be revealed that the same results were observed using the non-radioactive label such as biotin, compared with the use of the radioactive label.

As described above, the method of analyzing an XPG activity according to the present invention has the following advantages: 1) there is no cumbersome procedures such as overexpression and purification performed to prepare a recombinant protein, 2) it is inexpensive, 3) it is an analysis method that can be easily applied, 4) it can apply to various kinds of cell and tissue tests, 5) a DNA substrate is used at a small quantity, and 6) an analysis time is short. The XPG activity assay is very important in the fields of clinical diagnosis, development of novel drugs and cancer treatment. The method of analyzing an XPG activity according to the present invention can be useful in evaluating the XPG activity against the toxicity of surrounding environments such as UV radiation or chemical mutagens. Using the method of analyzing an XPG activity according to the present invention, the analysis may also be carried out using XPGs derived from various biological extracts while maintaining the reproducibility and sensitiveness of the analysis. Therefore, the analysis method according to the present invention may be used to evaluate the XPG activity of cells or tissues of interest at the same time.

The present invention provides an effective analysis method capable of quantitatively analyzing an XPG endonuclease activity of cells or tissues. According to the present invention, the analysis method has advantages in that the XPG endonuclease activity can be simply and cheaply performed without undergoing overexpression or purification of a recombinant protein, the XPG endonuclease activity of the cells or tissues can be analyzed at a level close to the actual organism activity even when the DNA substrate is used at a relatively small amount, and an analysis time can be shortened. Furthermore, the kit for quantitatively analyzing an XPG endonuclease activity according to the present invention can be used for clinical diagnosis to confirm the presence of DNA damage, or used to screen therapeutic agents for treating cancer-prone syndromes, skin cancer, etc.

While the invention has been shown and described with reference to certain exemplary embodiments thereof, it will be understood by those skilled in the art that various changes in form and details may be made therein without departing from the scope of the invention as defined by the appended claims.

What is claimed is:

1. A method of analyzing an XPG endonuclease activity, comprising:
preparing a biological extract sample including XPG (xeroderma pigmentosum (XP) of the complementation group G);
preparing a DNA bubble substrate;
forming the DNA bubble substrate whose 3′-terminus is labeled by attaching a detectable label to the 3′-terminus of the DNA bubble substrate; and
mixing the biological extract sample and the DNA bubble substrate whose 3′-terminus is labeled.

2. The method according to claim 1, wherein the biological extract sample is a cellular nuclear extract, a total cellular protein extract or a total tissue protein extract.

3. The method according to claim 1, wherein the DNA bubble substrate has oligonucleotides set forth in SEQ ID NO: 1 and SEQ ID NO: 2 which complementarily bind to each other.

4. The method according to claim 1, wherein the detectable label is a radioactive isotope, a fluorescent material, a luminescent material or a precursor of the luminescent material.

5. The method according to claim 4, wherein the detectable label is selected from the group consisting of ³²P, ³⁵S, ¹³¹I, ¹²³I, ¹²⁵I, ³H, carboxyfluorescein (FAM), tetramethylrhodamine (TAMRA), Cy3, Cy5, IRDye series, fluorescein, fluorescein isothiocyanate (FITC), rhodamine, Texas Red, Alexa series, digoxigenin (DIG) and biotin.

6. The method according to claim 4, further comprising, after the mixing of the biological extract sample and the DNA bubble substrate whose 3′-terminus is labeled:
determining whether the DNA bubble substrate is incised.

7. The method according to claim 6, wherein the incised DNA bubble has a size of 30 or less nucleotides.

8. The method according to claim 1, wherein the mixing of the biological extract sample and the DNA bubble substrate is performed in the presence of a reaction buffer.

9. The method according to claim 8, wherein the reaction buffer has a pH value of 6.0 to 8.5.

10. The method according to claim 8, wherein the reaction buffer includes MgCl₂ or MnCl₂.

11. The method according to claim 10, wherein an amount of the added MgCl₂ or MnCl₂ is in a range of 2 to 10 mM.

12. A kit for analyzing an XPG endonuclease activity, comprising the biological extract sample and the DNA bubble substrate defined in claim 1.


SEQUENCE LISTING (2 sequences)

SEQ ID NO: 1 - DNA, 90 bp, Artificial Sequence
  Oligonucleotide Substrate Upper Strand
ccagtgatcacatacgctttgctaggacatcccccccccccccccccccccccccccccccagtgccacgttgtatgcccacgttgaccg

SEQ ID NO: 2 - DNA, 90 bp, Artificial Sequence
  Oligonucleotide Substrate Down Strand
cggtcaacgtgggcatacaacgtggcactgttttttttttttttttttttttttttttttatgtcctagcaaagcgtatgtgatcactgg